(19) United States
(12) Patent Application Publication
(10) Pub. No.: US 2019/0374656 A1
(43) Pub. Date: Dec. 12, 2019

(54) METHODS FOR MAINTAINING OPTIMUM DNA METHYLATION BY ENDOGENOUS METHYLATION AND DEMETHYLATION OF DNA

(71) Applicant: Aether Therapeutics Inc., Calgary (CA)

(72) Inventor: Bradley G. THOMPSON, Calgary (CA)

(21) Appl. No.: 16/005,338

(22) Filed: Jun. 11, 2018

(51) Int. Cl. A61K 48/00; A61K 45/06; C12N 7/00

(52) U.S. Cl. CPC A61K 48/0058; C12N 7/00; A61K 45/06

(57) ABSTRACT

The present disclosure relates to one or more agents, therapies, treatments, and methods of use of the agents and/or therapies and/or treatments for endogenously increasing or decreasing DNA methylation in two or more genes in one or more tissues. Embodiments of the present disclosure can be used as a therapy or a treatment for a subject that has a condition such as aging, addiction, drug tolerance, and other diseases.

### TECHNICAL FIELD

The present disclosure relates to agents, therapies, and methods of use of compounds, agents and/or therapies, for increasing and/or decreasing DNA methylation of two or more genes and/or tissues. In particular, embodiments of the present disclosure relate to the use of such agents, therapies and methods as a therapy or treatment for conditions resulting from hypo-methylation and/or hyper-methylation of DNA.

### BACKGROUND

Upon fertilization, zygotes undergo a general de-methylation of deoxyribonucleic acid (DNA). As the zygote divides and differentiates, re-methylation of the DNA occurs and new methyl groups are added to previously un-methylated DNA.

Conditions such as aging, disease, drug tolerance, and addiction lead to both upregulation and/or downregulation of numerous genes in various tissues. This upregulation and/or down regulation may be the result of either hypo-methylation of one specific gene and/or hyper-methylation of another specific gene. For example, genes that are hyper-methylated are often downregulated, and as a result, the products of gene transcription and translation are reduced. In contrast, genes that are hypo-methylated are often upregulated, and as a result, the products of gene transcription and translation are increased.

Known approaches for the methylation of DNA with exogenous agents include treatment with chemotherapeutic agents, which are commonly used to treat cancers, and azidothymidine (AZT).

Experimental approaches for targeted methylation of DNA include use of modified clustered regularly interspaced short palindromic repeats (CRISPR) techniques.

Known approaches for the de-methylation of DNA with exogenous agents include treatment with cytidine analogues such as 5-azacytidine, and local anesthetic agents, such as procaine and lidocaine.

Experimental approaches for targeted de-methylation of DNA include gene therapy with plasmids encoding for TALE-TET1 fusion proteins and use of modified CRISPR techniques.

The methylation and de-methylation of multiple genes to restore the DNA methylation levels of a tissue or tissues to those found prior to the onset of a condition or to a desired level may be useful in treating conditions such as aging, addiction, drug tolerance, and disease.

### SUMMARY

Some embodiments of the present disclosure relate to a method of making an agent/target cell complex. The method comprises a step of administering a therapeutically effective amount of the agent to a subject, wherein in some embodiments of the present disclosure the agent/target cell complex causes the methylation and/or de-methylation of two or more genes in one or more tissues of the subject.

In some embodiments of the present disclosure at least two agents may be administered to a subject to form at least two different types of agent/target cell complexes and each type of agent/target cell complex causes the methylation and/or de-methylation of at least two genes in one or more tissues of the subject.

In some embodiments of the present disclosure at least three agents may be administered to a subject to form at least three different types of agent/target cell complexes and each type of agent/target cell complex causes the methylation and/or de-methylation of at least two genes in one or more tissues of the subject.

In some embodiments of the present disclosure at least four agents may be administered to a subject to form at least four different types of agent/target cell complexes and each type of agent/target cell complex causes the methylation and/or de-methylation of at least two genes in one or more tissues of the subject.

In some embodiments of the present disclosure at least five agents may be administered to a subject to form at least five different types of agent/target cell complexes and each type of agent/target cell complex causes the methylation and/or de-methylation of at least two genes in one or more tissues of the subject.

In some embodiments of the present disclosure at least six agents may be administered to a subject to form at least six different types of agent/target cell complexes and each type of agent/target cell complex causes the methylation and/or de-methylation of at least two genes in one or more tissues of the subject.

In some embodiments of the present disclosure at least seven agents may be administered to a subject to form at least seven different types of agent/target cell complexes and each type of agent/target cell complex causes the methylation and/or de-methylation of at least two genes in one or more tissues of the subject.

In some embodiments of the present disclosure at least eight agents may be administered to a subject to form at least eight different types of agent/target cell complexes and each type of agent/target cell complex causes the methylation and/or de-methylation of at least two genes in one or more tissues of the subject.

Some embodiments of the present disclosure relate to a method of making an agent/target cell complex, the method comprising a step of administering a sufficient amount of an agent to a target cell whereby the agent/target cell complex is formed. Wherein in some embodiments of the present disclosure the agent/target cell complex causes the causes the methylation and/or de-methylation of two or more genes in one or more tissues. In some embodiments of the present disclosure the agent/target cell complex causes the endogenous production of one or more regulatory molecules that causes the methylation and/or de-methylation of two or more genes in one or more tissues. In some embodiments of the present disclosure the agent/target cell complex causes the endogenous production of both of the one or more antagonists and the one or more regulatory molecules that causes the methylation and/or de-methylation of two or more genes.

Some embodiments of the present disclosure relate to a pharmaceutical composition that comprises an agent, a pharmaceutically acceptable carrier, and/or an excipient. In some embodiments of the present disclosure the agent causes the methylation and/or de-methylation of two or more genes. In some embodiments of the present disclosure the agent causes the endogenous production of one or more regulatory molecules that causes the targeted methylation and/or de-methylation of two or more genes. In some embodiments of the present disclosure the agent causes the endogenous production of both of the one or more antagonists and the one or more regulatory molecules that causes the methylation and/or de-methylation of two or more genes in one or more tissues.

Some embodiments of the present disclosure relate to a kit used for treatment of a condition or for delivery of a therapy to a subject. The kit comprises a unit dosage of an agent, a carrier for the unit dosage, and instructions for administering the unit dosage to the subject. Wherein in some embodiments of the present disclosure the agent causes the methylation and/or de-methylation of two or more genes in one or more tissues. Wherein in some embodiments of the present disclosure the agent causes the endogenous production of one or more regulatory molecules that causes the methylation and/or de-methylation of two or more genes in one or more tissues. Wherein in some embodiments of the present disclosure the agent causes the endogenous production of both of the one or more antagonists and the one or more regulatory molecules that causes the methylation and of de-methylation of two or more genes in one or more tissues. The carrier may be a solid carrier, such as a pill or tablet, or a liquid. The instructions may describe how the solid carrier may be administered to a subject for an optimal effect. The instructions may also describe how the liquid carrier may be administered to a subject by various routes of administration.

Some embodiments of the present disclosure relate to a method of treating a condition. The method comprises a step of administering to a subject a therapeutically effective amount of an agent that may cause one, or some, or all of the methylation and/or de-methylation of two or more genes in one or more tissues.

Some embodiments of the present disclosure relate to a method for causing the endogenous production of one, or some or all, of: an antagonist of or a regulatory molecule that inhibits the methylation process and/or de-methylation process that affects two or more genes in one or more tissues.

Some embodiments of the present disclosure relate to at least one approach for the methylation and/or de-methylation of two or more genes in one or more tissues. A first approach utilizes one or more gene vectors that contain nucleotide sequences and/or genes that cause a subject that receives the one or more gene vectors to produce, or increase production of, one or more agonists or antagonists to cause the methylation and/or de-methylation of two or more genes in one or more tissues. Another approach utilizes one or more gene vectors that contain nucleotide sequences and/or genes that cause a subject that receives the one or more gene vectors to produce, or increase production of, one or more regulatory molecules that cause the methylation and/or de-methylation of two or more genes in one or more tissues. Another approach utilizes one or more gene vectors that contain nucleotide sequences and/or genes that cause the subject that receives the one or more gene vectors to produce, or increase production of, the one or more antagonists and the one or more regulatory molecules.

Without being bound by any particular theory, therapies or treatments that comprise the use of antagonists or agonists of the targeted methylation or demethylation of a single gene, or the use of viral vectors that cause the targeted methylation or demethylation of a single gene, may be limited by the effectiveness of the treatment or therapy to access the subject's affected cells and/or tissues. Furthermore, the antagonists or agonists may impact only one of many genes found in the cell and/or tissues, which means that other hyper-methylated and/or hypo-methylated genes may still influence the subject's condition.

Embodiments of the present disclosure may be useful for treating conditions such as aging, addiction, drug tolerance, and disease, where the methylation and de-methylation of multiple genes in one or more tissues may cause the condition.

### DETAILED DESCRIPTION

### Definitions

Unless defined otherwise, all technical and scientific terms used herein have the meanings that would be commonly understood by one of skill in the art in the context of the present description. Although any methods and materials similar or equivalent to those described herein can also be used in the practice or testing of the present disclosure, the preferred methods and materials are now described. All publications mentioned herein are incorporated herein by reference to disclose and describe the methods and or materials in connection with which the publications are cited.

As used herein, the singular forms “a”, “an”, and “the” include plural references unless the context clearly dictates otherwise. For example, reference to “an agent” includes one or more agents and reference to “a subject” or “the subject” includes one or more subjects.

As used herein, the terms “about” or “approximately” refer to within about 25%, preferably within about 20%, preferably within about 15%, preferably within about 10%, preferably within about 5% of a given value or range. It is understood that such a variation is always included in any given value provided herein, whether or not it is specifically referred to.

As used herein, the term “agent” refers to a substance that, when administered to a subject, causes one or more chemical reactions and/or one or more physical reactions and/or or one or more physiological reactions and/or one or more immunological reactions in the subject.

As used herein, the term “antagonist” refers to an agent that can, directly or indirectly, inhibit a physiologic activity and/or production of a target molecule within a subject that receives the agent.

As used herein, the term “ameliorate” refers to improve and/or to make better and/or to make more satisfactory.

As used herein, the term “biomolecule” refers to a carbohydrate, a protein, an amino acid sequence, a nucleic acid, a lipid, a primary metabolite, a secondary metabolite that is found within a subject. A biomolecule may be endogenous or exogenous.

As used herein, the term “cell” refers to a single cell as well as a plurality of cells or a population of the same cell type or different cell types. Administering an agent to a cell includes in vivo, in vitro and ex vivo administrations or combinations thereof.

As used herein, the term “complex” refers to an association, either direct or indirect, between one or more particles of an agent and one or more target cells. This association results in a change in the metabolism of the target cell. As used herein, the phrase “change in metabolism” refers to an increase or a decrease in the one or more target cells' production of deoxyribonucleic acid (DNA), ribonucleic acid (RNA), one or more proteins, or any post-translational modifications of one or more proteins.

As used herein, the terms “de-methylation molecule” and “de-methylation molecules” refer to one or more molecules that directly or indirectly cause the removal of one or more methyl groups from a portion of DNA.

As used herein, the term “effector molecule” refers to a molecule within a subject that can directly or indirectly regulate the metabolic activity of a target cell by increasing or decreasing the production of DNA, RNA, amino-acid sequences and/or by increasing or decreasing any post-translational modifications of one or more proteins.

As used herein, the term “endogenous” refers to the synthesis, production and/or modification of a molecule that originates within a subject.

As used herein, the term “excipient” refers to any substance, not itself an agent, which may be used as a component within a pharmaceutical composition or a medicament for administration of a therapeutically effective amount of the agent to a subject. Additionally or alternatively an excipient may alone, or in combination with further chemical components, improve the handling and/or storage properties, and/or permit or facilitate formation of a dose unit, of the agent. Excipients include, but are not limited to, one or more of: a binder, a disintegrant, a diluent, a buffer, a taste enhancer, a solvent, a thickening agent, a gelling agent, a penetration enhancer, a solubilizing agent, a wetting agent, an antioxidant, a preservative, a surface active agent, a lubricant, an emollient, a substance that is added to mask or counteract a disagreeable odor, fragrances or taste, a substance that is added to improve appearance or texture of the composition and a substance used to form the pharmaceutical compositions or medicaments. Any such excipients can be used in any dosage forms according, to the present disclosure. The foregoing classes of excipients are not meant to be exhaustive but are provided merely as illustrative of what a person of skill in the art would know and would also recognize that additional types and combinations of excipients may be used to achieve delivery of a therapeutically effective amount of the agent to a subject through one or more routes of administration.

As used herein, the term “exogenous” refers to a molecule that is within a subject but that did not originate within the subject.

As used herein, the terms “inhibit”, “inhibiting”, and “inhibition” refer to a decrease in activity, response, or other biological parameter of a biologic process, disease, disorder or symptom thereof. This can include but is not limited to the complete ablation of the activity, response, condition, or disease. This may also include, for example, a 10% reduction in the activity, response, condition, or disease as compared to the native or control level. Thus, the reduction can be a 10%, 20%, 30%, 40%, 50%, 60%, 70%, 80%, 90%; 100%, or any amount of reduction in between the specifically recited percentages, as compared to native or control levels.

As used herein, the term “medicament” refers to a medicine and/or pharmaceutical composition that comprises the agent and that can promote recovery from a disease, disorder or symptom thereof and/or that can prevent a disease, disorder or symptom thereof and/or that can inhibit the progression of a disease, disorder, or symptom thereof.

As used herein, the terms “methylation molecule” and “methylation molecules” refer to one or more molecules that directly or indirectly cause the addition of one or more methyl groups to a portion of DNA.

As used herein, the term “patient” refers to a subject that is afflicted with an infectious disease. The term “patient” includes human and veterinary subjects.

As used herein, the term “pharmaceutical composition” means any composition for administration of an agent to a subject in need of therapy or treatment of a disease, disorder or symptom thereof. Pharmaceutical compositions may include additives such as pharmaceutically acceptable carriers, pharmaceutically accepted salts, excipients and the like. Pharmaceutical compositions may also additionally include one or more further active-ingredients such as antimicrobial agents, anti-inflammatory agents, anesthetics, analgesics, and the like.

As used herein, the term “pharmaceutically acceptable carrier” refers to an essentially chemically inert and nontoxic component within a pharmaceutical composition or medicament that does not inhibit the effectiveness and/or safety of the agent. Some examples of pharmaceutically acceptable carriers and their formulations are described in Remington (1995, The Science and Practice of Pharmacy (19th ed.) ed. A. R. Gennaro, Mack Publishing Company, Easton, Pa.), the disclosure of which is incorporated herein by reference. Typically, an appropriate amount of a pharmaceutically acceptable carrier is used in the formulation to render the formulation isotonic. Examples of suitable pharmaceutically acceptable carriers include, but are not limited to: saline solutions, glycerol solutions, ethanol, N-(1(2, 3-dioleyloxy)propyl)-N,N,N-trimethylammonium chloride (DOTMA), diolesylphosphotidylethanolamine (DOPE), and liposomes. Such pharmaceutical compositions contain a therapeutically effective amount of the agent, together with a suitable amount of one or more pharmaceutically acceptable carriers and/or excipients so as to provide a form suitable for proper administration to the subject. The formulation should suit the route of administration. For example, oral administration may require enteric coatings to protect the agent from degrading within portions of the subject's gastrointestinal tract. In another example, injectable routes of administration may be administered in a liposomal formulation to facilitate transport throughout a subject's vascular system and to facilitate delivery across cell membranes of targeted intracellular sites.

As used herein, the phrases “prevention or” and “preventing” refer to avoiding an onset or progression of a disease, disorder, or a symptom thereof.

As used herein, the terms “production”, “producing” and “produce” refer to the synthesis and/or replication of DNA, the transcription of one or more sequences of RNA, the translation of one or more amino acid sequences, the post-translational modifications of amino acid sequences, the synthesis or altered functionality of one or more regulatory molecules that can influence the production or functionality of an effector molecule or an effector cell.

As used herein, the terms “promote”, “promotion”, and “promoting” refer to an increase in an activity, response, condition, disease process, or other biological parameter. This can include but is not limited to the initiation of the activity, response, condition, or disease process. This may also include, for example, a 10% increase in the activity, response, condition, or disease as compared to the native or control level. Thus, the increase in an activity, response, condition, disease, or other biological parameter can be a 10%, 20%, 30%, 40%, 50%, 60%, 70%, 80%, 90%, 100%, or more, including any amount of increase in between the specifically recited percentages, as compared to native or control levels.

As used herein, the term “prophylactic administration” refers to the administration of any composition to a subject, in the absence of any symptom or indication of a disease or disorder, to prevent the occurrence of and/or the progression of the disease or disorder within the subject.

As used herein, the terms “regulation of methylation” and “regulating of methylation” both refer to a process whereby a portion of DNA is methylated or de-methylated based upon the actions, direct or indirect, of a methylation molecule or a de-methylation molecule, respectively.

As used herein, the terms “signal molecule”, “signaling molecule” and “regulatory molecule” can be used interchangeably and refer to a molecule that can directly or indirectly affect the production and/or functionality of an effector molecule or effector cell. Signal molecules can be enzymes or other types of biomolecules and they can act as a direct ligand on a target cell or they may influence the levels or functionality of a downstream ligand or receptor for a ligand.

As used herein, the term “subject” refers to any therapeutic target that receives the agent. The subject can be a vertebrate, for example, a mammal including a human. The term “subject” does not denote a particular age or sex. The term “subject” also refers to one or more cells of an organism, an in vitro culture of one or more tissue types, an in vitro culture of one or more cell types; ex vivo preparations; and a sample of biological materials such as tissue and/or biological fluids.

As used herein, the term “large cell” refers to one or more cells and/or cell types that are deleteriously affected, either directly or indirectly, by a disease.

As used herein, the term “therapeutically effective amount” refers to the amount of the agent used that is of sufficient quantity to ameliorate, treat and/or inhibit one or more of a disease, disorder or a symptom thereof. The “therapeutically effective amount” will vary depending on the agent used, the route of administration of the agent and the severity of the disease, disorder or symptom thereof. The subject's age, weight and genetic make-up may also influence the amount of the agent that will be a therapeutically effective amount.

As used herein, the terms “treat”, “treatment” and “treating” refer to obtaining a desired pharmacologic and/or physiologic effect. The effect may be prophylactic in terms of completely or partially preventing an occurrence of a disease, disorder or symptom thereof and/or the effect may be therapeutic in providing a partial or complete amelioration or inhibition of a disease, disorder, or symptom thereof. Additionally, the term “treatment”, refers to any treatment of a disease, disorder, or symptom thereof in a subject and includes: (a) preventing the disease from occurring in a subject which may be predisposed to the disease but has not yet been diagnosed as having it; (b) inhibiting the disease; and (c) ameliorating the disease.

As used herein, the terms “unit dosage form” and “unit dose” refer to a physically discrete unit that is suitable as a unitary dose for patients. Each unit contains a predetermined quantity of the agent and optionally, one or more suitable pharmaceutically acceptable carriers, one or more excipients, one or more additional active-ingredients, or combinations thereof. The amount of agent within each unit is a therapeutically effective amount.

In one embodiment of the present disclosure, the pharmaceutical compositions disclosed herein comprise an agent as described above in a total amount by weight of the composition of about 0.1% to about 2%. For example, the amount of the agent by weight of the pharmaceutical composition may be about 0.1%, about 0.2%, about 0.3%, about 0.4%, about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, or about 2%.

Where a range of values is provided herein, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also, encompassed within the disclosure, subject to any specifically excluded limit in the stated range. Where the stated range includes one or both of the limits, ranges excluding either or both of those included limits are also included in the disclosure.

The present disclosure relates to one or more agents, therapies, treatments, and methods of use of the agents and/or therapies and/or treatments that cause the methylation and/or de-methylation of two or more genes. Some embodiments of the present disclosure relate to methods for making a complex between at least one particle of an agent and at least one target cell of a subject where the complex causes the methylation and/or de-methylation of two or more genes in one or more tissues.

Embodiments of the present disclosure can be used as a therapy or a treatment for a patient.

In some embodiments of the present disclosure, the condition may be one that relates to an altered regulation of methylation and/or demethylation of two or more genes within the subject that has the condition, as compared to prior to the onset of the condition. Some non-limiting examples of such a condition may include: cancer, autoimmune disease, a dermatological condition, cardiovascular disease, fibrotic disease, diabetes, addiction, drug tolerance, skin and degenerative neural-diseases.

In some embodiments of the present disclosure, the agent can be administered to the subject by an intravenous route, an intramuscular route, an intraperitoneal route, an intrathecal route, an intravesical route, a topical route, an intranasal route, a transmucosal route, a pulmonary route, or combinations thereof.

In some embodiments of the present disclosure, the agent can be administered to the subject by pipetting a dose of the agent into an in vitro cell culture; perfusing or immersing an ex vivo cell or tissue preparation with a solution that comprises the agent; mixing a biological fluid sample with a solution or substrate that comprises the agent, or combinations thereof.

Some embodiments of the present disclosure relate to an agent that can be administered to a subject with the condition. When a therapeutically effective amount of the agent is administered to the subject, the agent may change the methylation or de-methylation of two or more genes in one or more tissues.

For example, the agent may increase the methylation and/or de-methylation of two or more genes by changing the production of one or more sequences of DNA, one or more sequences of RNA and/or one or more proteins and/or one or more regulatory molecules that regulate the subject's levels and/or functionality of methylation/demethylation molecules and/or methylation/de-methylation effector molecules.

In some embodiments of the present disclosure, the subject may respond to receiving a therapeutically amount of the agent by changing production and/or functionality of one or more intermediary molecules by changing production of one or more DNA sequences, one or more RNA sequences, and/or one or more proteins that regulate the levels and/or functionality of the one or more intermediary molecules. The one or more intermediary molecules regulate the subject's levels and/or functionality of the one or more of the subject's levels and/or functionality of methylation/demethylation molecules and/or methylation/demethylation effector molecules.

In some embodiments of the present disclosure, administering a therapeutic amount of the agent to a subject changes the production, functionality or both of one or more regulatory molecules that inhibits the production or functionality of one or more methylation/de-methylation molecules. The one or more regulatory molecules can be a sequence of DNA, RNA or amino acids that inhibits the production and/or functionality of one or more one or more methylation/de-methylation molecules after administration of the agent. The agent can increase the production and/or functionality of the one or more regulatory molecules by increasing one or more of synthesis of one or more nucleotides, nucleosides, sequences or genes that are related to causing increased amounts or functionality of the one or more regulatory molecules; transcription of RNA that is related to increased amounts or functionality of the one or more regulatory molecules; or translation of one or more amino acids or amino acid sequences that cause increased amounts or functionality of the one or more regulatory molecules.

In some embodiments of the present disclosure, the agent can be: a vector used for gene therapy; one or more selected nucleotides; a sequence of nucleotides; one or more nucleosides; a sequence of nucleosides; a DNA complex; one or more amino acids; a sequence of ammo acids; a live microorganism; an attenuated microorganism; a dead microorganism; a recombinant virus; a non-recombinant virus; or combinations thereof.

In some embodiments of the present disclosure, the agent is a gene vector used for gene therapy. The gene therapy is useful for increasing and/or decreasing the production of one or more antagonists or agonists that inhibit or increase the production or functionality one or more methylation/de-methylation molecules. Additionally or alternatively, the gene therapy is useful for inhibiting or for increasing the production of one or more regulatory molecules that inhibit or increase the production or functionality one or more methylation/de-methylation molecules.

In some embodiments of the present disclosure, the gene vector is a virus that can be within one or more of the following genus: flavivirus, influenza, enterovirus, rotavirus, rubellavirus, rubivirus morbillivirus, orthopoxvirus, varicellovirus, dependoparvovirus, alphabaculovirus, betabaculovirus, deltabaculovirus, gammabaculovirus, mastadenovirus, simplexvirus, varicellovirus, cytomegalovirus, or combinations thereof. In some embodiments of the present disclosure the virus is an attenuated virus.

The embodiments of the present disclosure also relate to administering a therapeutically effective amount of the agent. The therapeutically effective amount of the agent will not substantially increase or cause any deleterious conditions within the subject. For example, the therapeutically effective amount will not cause cytokinesis, hypercytokinemia, or any other uncontrolled, or partially controlled, upregulation of the subject's immune system. In some embodiments of the present disclosure, the therapeutically effective amount of the agent that is administered to a patient is between about 10 and about 1×10¹⁶ TCID₅₀/kg (50% tissue culture infective dose per kilogram of the patient's body weight). In some embodiments of the present disclosure the therapeutically effective amount of the agent that is administered to the patient is about 1×10¹³ TCID₅₀/kg. In some embodiments of the present disclosure, the therapeutically effective amount of the agent that is administered to a patient is measured in TPC/kg (total particle count of the agent per kilogram of the patient's body weight). In some embodiments the therapeutically effective amount of the agent is between about 10 and about 1×10¹⁶ TCP/kg.

Some embodiments of the present disclosure relate to a method for making an agent/target cell complex within a subject. The method comprises a step of administering a therapeutically effective amount of the agent to the subject. The complex comprises at least one particle of an agent and one or more target cells. When the complex is formed, it affects a change in metabolism of the one or more target cells that results in the subject down or upregulating the production and/or functionality of one or more methylation/de-methylation molecules.

Some examples of a target cell that can form the agent/target cell complex include, but are not limited to: an adrenal gland cell; a B cell; a bile duct cell; a cancer cell, a chondrocyte; a cochlear cell; a corneal cell; a dendritic cell, an endocardium cell; an endometrial cell; an endothelial cell; an epithelial cell; an eosinophil; a fibroblast; a hair follicle cell; a hepatocyte; a lymph node cell; a macrophage; a mucosal cell; a myocyte; a neuron; a glomeruli cell; an optic nerve cell; an osteoblast; an ovarian tissue cell; a pancreatic islet beta cell; a pericardium cell; a platelet; a red blood cell (RBC); a retinal cell; a scleral cell; a Schwann cell; a stem cell, a T cell; a testicular tissue cell; a thyroid gland cell; a uveal cell; or combinations thereof.

Some embodiments of the present disclosure relate to a therapy that can be administered to a subject with one or more conditions that are related to, directly or indirectly, an altered regulation of methylation and/or demethylation of two or more genes within the subject. The therapy comprises a step of administering to the subject a therapeutically effective amount of an agent that will decrease or increase production or activity of one or more regulatory molecules and/or one or more DNA methylation/de-methylation molecules. When the therapy is administered to a patient, the therapy will decrease the in vivo production and/or functionality of one or more regulatory molecules and/or one or more DNA methylation molecules and/or demethylation molecules. The decreased or increased production and/or functionality of the DNA methylation and/or de-methylation molecules may reduce or remove the deleterious effects of the condition upon the patient.

Some embodiments of the present disclosure relate to a method of treating a condition where the method comprises a step of administering to the subject a therapeutically effective amount of an agent that will decrease or increase production or activity of one or more regulatory molecules and/or one or more DNA methylation and/or de-methylation molecules.

Some embodiments of the present disclosure relate to therapies, treatments, and methods of use of more than one agent for the methylation and/or de-methylation of two or more genes in one or more tissues. For example, a therapy, treatment and method may comprise the use of: a first agent that can be used to methylate or de-methylate a gene: a second agent can be used to methylate or de-methylate a second gene; a third agent can be used to methylate or de-methylate a third gene. In some embodiments of the present disclosure there may be three, four, five, six, seven, or eight or more agents used to methylate or de-methylate three or more genes in one or more tissues.

Some embodiments of the present disclosure relate to one or more therapies, treatments, and methods of use of one or more of the first agent, the second agent, the third agent and another agent and/or further agents that inhibit the methylation or de-methylation of two more genes. The first agent, the second agent, the third agent and another agent and/or further agents can each increase the production and/or functionality of an antagonist or agonist of methylation and/or de-methylation for each of the agents' respective target genes.

In some embodiments of the present disclosure, the first agent, the second agent, the third agent and the another agent and/or further agents can each increase or decrease the production and/or functionality of a regulatory molecule that, directly or indirectly, inhibits the production and/or functionality of each of the agents use to methylate and/or de-methylate part of the DNA within a gene.

### EXAMPLES.

Some examples of regulatory molecules that are involved in regulation of melthylation include: enzymes such as DNA methyltransferase 1 (DNMT1), DNMT2, DNMT3, DNMT3a, DNMT3b, DNMT3L, ten-eleven translocation 5-mC hydroxylase 1 (TET1), TET2 and TET3.

1. A method of making an agent/target cell complex, the method comprising a step of administering a therapeutically effective amount of an agent to a subject, wherein the agent/target cell complex changes the subject's endogenous production and/or the functionality of an enzyme that is involved in regulating methylation.

2. A method of making an agent/target cell complex, the method comprising a step of administering a therapeutically effective amount of an agent to a subject, wherein the agent/target cell complex changes the subject's endogenous production and/or the functionality of an antagonist of an enzyme that is involved in regulation of methylation.

3. The method of claim 1 or claim 2, wherein the regulation of methylation is methylation.

4. The method of claim 1 or claim 2, wherein the regulation of methylation is de-methylation.

5. The method of claim 1, wherein the enzyme is one of a DNA methyltransferase, a ten-eleven translocation 5-mC hydroxylase and a combination thereof.

6. The method of claim 2, wherein the antagonist is one of a DNA methyltransferase antagonist, a ten-eleven translocation 5-mC hydroxylase antagonist and a combination thereof.

7. The method of claim 2, wherein the antagonist is a regulatory molecule.

8. The method of claim 1 or claim 2, wherein the agent is at least one of a gene vector used for gene therapy, one or more selected nucleotides, a sequence of nucleotides, one or more nucleosides, a sequence of nucleosides, a DNA complex, one or more amino acids, a sequence of amino acids, a live microorganism, an attenuated microorganism, a dead microorganism, a recombinant virus, a non-recombinant virus and a combination thereof.

9. The method of claim 1 or claim 2, wherein the agent/target cell complex comprises a target cell that is at least one of an adrenal gland cell; a B cell; a bile duct cell; a cancer cell, a chondrocyte; a cochlear cell; a corneal cell; a dendritic cell, an endocardium cell; an endometrial cell; an endothelial cell; an epithelial cell; an eosinophil; a fibroblast; a hair follicle cell; a hepatocyte; a lymph node cell; a macrophage; a mucosal cell; a myocyte; a neuron; a glomeruli cell; an optic nerve cell; an osteoblast; an ovarian tissue cell; a pancreatic islet beta cell; a pericardium cell; a platelet; a red blood cell (RBC); a retinal cell; a scleral cell; a Schwann cell; a stem cell, a T cell; a testicular tissue cell; a thyroid gland cell; and a uveal cell.

10. The method of claim 1 or claim 2, further comprising a step of administering a second agent that changes a production and/or a functionality of a second enzyme that is involved in regulating methylation.

11. The method of claim 10, wherein the second agent changes a production and/or a functionality of a regulatory molecule that changes the production and/or the functionality of the second enzyme.

12. The method of claim 10, further comprising a step of administering a third agent that changes a production and/or a functionality of a third enzyme that is involved in regulation of methylation.

13. The method of claim 12, wherein the third agent changes a production and/or functionality of a regulatory molecule that changes the production and/or the functionality of the third enzyme.

14. The method of claim 12, further comprising a step of administering a fourth agent that changes a production and/or a functionality of a fourth enzyme that is involved in the regulation of methylation.

15. The method of claim 14, wherein the fourth agent changes a production and/or functionality of a regulatory molecule that changes the production and/or the functionality of the fourth enzyme.

16. The method of claim 14, further comprising a step of administering a fifth agent that changes a production and/or a functionality of a fifth enzyme that is involved in the regulation of methylation.

17. The method of claim 16, wherein the fifth agent changes a production and/or functionality of a regulatory molecule that changes the production and/or the functionality of the fifth DNA methylation or de-methylation enzyme.

18. The method of claim 16, further comprising a step of administering a sixth agent that changes a production and/or a functionality of a sixth enzyme that is involved in regulation of methylation.

19. The method of claim 18, wherein the sixth agent changes a production and/or functionality of a regulatory molecule that changes the production and/or the functionality of the sixth enzyme.

20. The method of claim 18, further comprising a step of administering a seventh agent that changes a production and/or a functionality of a seventh enzyme that is involved in regulation of methylation.

21. The method of claim 20, wherein the seventh agent changes a production and/or functionality of a regulatory molecule that changes the production and/or the functionality of the seventh enzyme.

22. The method of claim 20, further comprising a step of administering an eighth agent that changes a production and/or a functionality of a eighth enzyme that is involved in regulation of methylation.

23. The method of claim 22, wherein the eighth agent increases a production and/or functionality of a regulatory molecule that changes the production and/or the functionality of the eighth enzyme.

24. A pharmaceutical composition comprising:
a. an agent that changes a production and/or functionality of one or more regulatory molecules that changes a production and/or a functionality of the one or more enzymes that are involved in regulation of methylation;
b. a pharmaceutically acceptable carver; and/or
c. air excipient.

25. The pharmaceutical composition of claim 24, wherein the one or more regulatory molecules are each an enzyme.

26. The pharmaceutical composition of claim 25, wherein the enzyme is one of one of DNA methyltransferase 1 (DNMT1), DNMT2, DNMT3, DNMT3a, DNMT3b, DNMT3L and combinations thereof.

27. The pharmaceutical composition of claim 25, wherein the enzyme is one of ten-eleven translocation 5-mC hydroxylase 1 (TET1), TET2, TET3 and combinations thereof.

28. The pharmaceutical composition of claim 24, further comprising a second agent that changes a production and/or a functionality of a second enzyme that is involved in regulation of methylation.

29. The pharmaceutical composition of claim 28, wherein the second agent changes a production and/or a functionality of a second regulatory molecule that changes the production and/or the functionality of the second enzyme.

30. The pharmaceutical composition of claim 28, further comprising a third agent that changes a production and/or a functionality of a third enzyme that is involved in regulation of methylation.

31. The pharmaceutical composition of claim 30, wherein the third agent changes a production and/or a functionality of a regulatory molecule that changes the production and/or the functionality of the third enzyme.

32. The pharmaceutical composition of claim 30, further comprising a fourth agent that changes a production and/or a functionality of a fourth enzyme that is involved in regulation of methylation.

33. The pharmaceutical composition of claim 32, wherein the fourth agent changes a production and/or a functionality of a regulatory molecule that changes the production and/or the functionality of the fourth enzyme.

34. The pharmaceutical composition of claim 32, further comprising a fifth agent that changes a production and/or a functionality of a fifth enzyme that is involved in regulation of methylation.

35. The pharmaceutical composition of claim 34, wherein the fifth agent changes a production and/or a functionality of a regulatory molecule that changes the production and/or the functionality of the fifth enzyme.

36. The pharmaceutical composition of claim 34, further comprising a step of administering a sixth agent that changes a production and/or a functionality of a sixth enzyme that is involved in regulation of methylation.

37. The pharmaceutical composition of claim 36, wherein the sixth agent changes a production and/or functionality of a regulatory molecule that changes the production and/or the functionality of the sixth enzyme.

38. The pharmaceutical composition of claim 36, further comprising a step of administering a seventh agent that changes a production and/or a functionality of a seventh enzyme that is involved in regulation of methylation.

39. The pharmaceutical composition of claim 38, wherein the seventh agent changes a production and/or functionality of a regulatory molecule that changes the production and/or the functionality of the seventh enzyme.

40. The pharmaceutical composition of claim 38, further comprising a step of administering an eighth agent that increases or decreases a production and/or a functionality of a eighth enzyme that is involved in regulation of methylation.

41. The pharmaceutical composition of claim 40, wherein the eighth agent changes a production and/or functionality of a regulatory molecule that increases or decreases the production and/or the functionality of the eighth enzyme.

42. The pharmaceutical composition according to claim 24, wherein the pharmaceutical composition is in a solid form or a liquid form.

43. A method of treating one or more of cancer, autoimmune disease, aging, cardiovascular disease, fibrotic disease, diabetes, addiction, drug resistance, a dermatological condition and a degenerative neural disease, the method comprising a step of administering to a subject a therapeutically effective amount of an agent that changes a production and/or a functionality of two or more enzymes that are each involved in regulation of methylation.

44. The method according to claim 43, wherein the agent changes a production and/or the functionality of three or more enzymes that are each involved in regulation of methylation.

45. The method according to claim 44, wherein the agent changes a production and/or the functionality of four or more enzymes that are each involved in regulation of methylation.

46. The method according to claim 45, wherein the agent changes a production and/or the functionality of five or more enzymes that are each involved in regulation of methylation.

47. The method according to claim 46, wherein the agent changes a production and/or the functionality of six or more enzymes that are each involved in regulation of methylation.

48. The method according to claim 47, wherein the agent changes a production and/or the functionality of seven or more enzymes that are each involved in regulation of methylation.

49. The method according to claim 48, wherein the agent changes a production and/or the functionality of eight or more enzymes that are each involved in regulation of methylation.

50. The method according to claim 43, wherein the step of administering occurs by at least one of an intravenous route, an intramuscular route, an intraperitoneal route, an intrathecal route, an intravesical route, a topical route, an intranasal route, a transmucosal route, and a pulmonary route.

51. The method according to claim 43, wherein each of the two or more methylation enzymes is DNA methyltransferase 1 (DNMT1). DNMT2, DNMT3, DNMT3a, DNMT3b and DNMT3L.

52. The method according to claim 43, wherein each of the two or more enzymes is ten-eleven translocation 5-mC hydroxylase 1 (TET1), TET2 and TET3.

53. The method according to claim 43, wherein the agent changes a production and/or the functionality of a regulatory molecule that changes the production of or the functionality of at least one of the two or more enzymes.

54. The method according to claim 43, wherein the regulatory molecule is a sequence of DNA and/or a sequence of RNA that changes production of at least one of the at least two enzymes.

55. The method according to claim 43, wherein the agent is a vector containing a gene for changing levels of at least one of the at least two enzymes.

56. The method according to claim 43, wherein the therapeutically effective amount is between about 10 to about 1×10¹⁶ TCID₅₀/kg of the subject's body weight.

57. The method according to claim 43, wherein the therapeutically effective amount is between about 10 to about 1×10¹⁶ total particles of the agent.
